(19) United States
(12) Patent Application Publication
(10) Pub. No.: US 2018/0318191 A1
(43) Pub. Date: Nov. 8, 2018

(54) Dental Composition and Method

(71) Applicant: PHOENIX DENTAL, INC., Fenton, MI (US)

(72) Inventors: Charles F. Cox, Fenton, MI (US); Bradley A. Cox, Fenton, MI (US); Jeffrey S. Cox, Fenton, MI (US)

(21) Appl. No.: 15/971,394

(22) Filed: May 4, 2018

Related U.S. Application Data
(60) Provisional application No. 62/501,157, filed on May 4, 2017.

(51) Int. Cl. A61K 8/365; A61K 8/34; A61K 8/20; A61K 8/90; A61Q 11/00

(52) U.S. Cl. CPC A61K 8/365; A61K 8/345; A61K 2800/884; A61K 8/90; A61Q 11/00; A61K 8/20

(57) ABSTRACT

A dental cleaning system and method is provided. The dental system contains: a first component containing an aqueous composition that includes —a carboxylic acid, a glycol, a metal chloride hydrate, water, and a surfactant; and, a second optional component selected from a metal carbonate, a metal bicarbonate, and mixtures thereof. The dental cleaning method includes: applying component one to teeth to be cleaned to thereby create a film over the teeth; optionally applying component two over the film created by component one and reacting the two components; and rinsing the teeth once cleaning is affected.

### CROSS-REFERENCE TO RELATED APPLICATIONS

This application claims the benefit of U.S. Provisional Application Ser. No. 62/501,157 having a filing date of May 4, 2017, the contents of which are herein incorporated by reference.

### TECHNICAL FIELD

The present invention relates to dental hygiene, and more specifically, to compositions that enhance the efficacy and safety of dental cleaning compositions and dental cleaning methods.

### BACKGROUND OF THE INVENTION

An ongoing challenge in the art of dental hygiene, maintenance, and oral health includes the efficient cleaning of a patient's teeth, while yet reducing the wear and tear on the teeth and surrounding tissue. Oftentimes, cleaning requires scraping of the teeth both above and below the gum-lines. Stated another way, supragingival calculus is known to accumulate on the tooth crown above the gum line, whereas sub-gingival calculus occurs below the gum line along the teeth roots. Calculus is a mineralized mass that forms from recurring thin oral micro biofilm, typically composed of many different colonies of bacteria. When day-to-day home dental care is not sufficient, the calculus must be periodically removed to ensure that cavities do not occur and that the integrity of the teeth as well as the periodontal health is enhanced. During this process, the gums may become irritated depending on the starting condition of the gums at the beginning of the cleaning process. Furthermore, oftentimes bone deterioration occurs with the accumulation of calculus beneath the gum line. As a result, it is necessary to accommodate or encourage the healing and formation of the bone as well as preserving the teeth. One naturally-occurring healing and bone-forming agent is known as bone morphogenetic protein, which is typically produced by the body.

Another type of cleaning includes vibratory ultrasonic power-assisted cleaning methods. A concern with this type of cleaning is that these methods typically cause scarring and gouging of the tooth root surface as well as damage to the thin cementum that normally protects the root dentine. Accordingly, although associated power devices remove calculus, they may leave undesirable cracks, nooks, and crannies (or uneven profiles), on the surface of the teeth. These uneven profiles may potentially exacerbate the hygienic situation by increasing the propensity for bacterial flora to thrive on these uneven surfaces or profiles, which permits recurrent decay.

Gel-like cleaning aids, including fluoride varnishes, ether-containing agents, and chloroform-containing agents are sometimes painted on, but they are difficult to consistently apply across the tooth surface. Furthermore, these types of thicker cleaning aids are detrimental to the soft gum tissue surrounding the teeth. Yet another disadvantage with gel-type cleaning aids includes an insufficient holding or retention time on the tooth. Over time, saliva acts to flush the gel from the surface of the tooth, thereby reducing the calculus-removing efficacy of the cleaning aid.

A system and method of cleaning and tooth cleaning composition that addresses these concerns would therefore be an improvement in the art.

### SUMMARY OF THE INVENTION

The above concerns are reconciled by a dental system containing: a first component and a second optional component. The first component contains a composition containing an anhydrous carboxylic acid provided at about 7-19 weight percent, a glycol provided at about 10.0-20.0 weight percent, a metal chloride hydrate provided at about 2.0-5.0 weight percent, water provided at about 55.0-65.0 weight percent, and a surfactant provided at about 10.0-20.0 weight percent. The second optional component is selected from a metal carbonate, a metal bicarbonate, and mixtures thereof.

A dental cleaning method also provided in accordance with the present invention contains the steps of: providing a dental system as described above; preparing the site and teeth to be cleaned in a conservative manner; applying component one over the teeth to be cleaned, to create a film; applying component two over the film created by component one; allowing the contact of component one and component two to result in an effervescence over the teeth to be cleaned; and rinsing component one, component two, and any resulting debris from the teeth.

### DETAILED DESCRIPTION OF THE PREFERRED EMBODIMENTS

In accordance with the present invention, the present compositions have been found to be useful in cleaning the teeth, and more specifically, useful in rapidly removing, lifting, softening, and otherwise cleansing the calculus and debris from the tooth surface.

A first component (component one) of the overall composition, is an aqueous composition, and in accordance with the present invention, contains a carboxylic acid selected from the groups containing, but not limited to, organic acids containing one or more carboxyl groups, dicarboxylic acids, and mixtures thereof. The carboxylic acid may be provided as an anhydrous compound in a weight percent of about 3-35 weight percent, and more preferably 5-20 weight percent, and even more preferably 7-13 weight percent of the total composition. The carboxylic acid constituent of the present invention may, for example, be selected from citric acid, 2-hydroxy-1,2,3-propanetricarboxylic acid (IUPAC name 2-hydroxypropane-1,2,3-tricarboxylic acid), and/or one or more of the following:

Straight-chain, saturated carboxylic acids

| Carbon | Common | | Chemical | |
|---|---|---|---|---|
| atoms | name | IUPAC name | formula | Common location or use |
| 1 | Carbonic acid | Carbonic acid | OHCOOH | Blood and tissues |
| | | | | (bicarbonate buffer system) |
| 1 | Formic acid | Methanoic acid | HCOOH | Insect stings |
| 2 | Acetic acid | Ethanoic acid | CH₃COOH | Vinegar |
| 3 | Propionic acid | Propanoic acid | CH₃CH₂COOH | Preservative for stored |
| | | | | grains, body odor |
| 4 | Butyric acid | Butanoic acid | CH₃(CH₂)₂COOH | Butter |
| 5 | Valeric acid | Pentanoic acid | CH₃(CH₂)₃COOH | Valerian |
| 6 | Caproic acid | Hexanoic acid | CH₃(CH₂)₄COOH | Goat fat |
| 7 | Enanthic acid | Heptanoic acid | CH₃(CH₂)₅COOH | |
| 8 | Caprylic acid | Octanoic acid | CH₃(CH₂)₆COOH | Coconuts and breast milk |
| 9 | Pelargonic | Nonanoic acid | CH₃(CH₂)₇COOH | Pelargonium |
| | acid | | | |
| 10 | Capric acid | Decanoic acid | CH₃(CH₂)₈COOH | Coconut and Palm kernel oil |
| 11 | Undecylic | Undecanoic acid | CH₃(CH₂)₉COOH | |
| | acid | | | |
| 12 | Lauric acid | Dodecanoic acid | CH₃(CH₂)₁₀COOH | Coconut oil and hand wash |
| | | | | soaps |
| 13 | Tridecylic acid | Tridecanoic acid | CH₃(CH₂)₁₁COOH | |
| 14 | Myristic acid | Tetradecanoic | CH₃(CH₂)₁₂COOH | Nutmeg |
| | | acid | | |
| 15 | Pentadecylic | Pentadecanoic | CH₃(CH₂)₁₃COOH | |
| | acid | acid | | |
| 16 | Palmitic acid | Hexadecanoic | CH₃(CH₂)₁₄COOH | Palm oil |
| | | acid | | |
| 17 | Margaric acid | Heptadecanoic | CH₃(CH₂)₁₅COOH | |
| | | acid | | |
| 18 | Stearic acid | Octadecanoic | CH₃(CH₂)₁₆COOH | Chocolate, waxes, soaps, and |
| | | acid | | oils |
| 19 | Nonadecylic | Nonadecanoic | CH₃(CH₂)₁₇COOH | Fats, vegetable |
| | acid | acid | | oils, pheromone |
| 20 | Arachidic acid | Icosanoic acid | CH₃(CH₂)₁₈COOH | Peanut oil |

Other carboxylic acids

| Compound class | Members |
|---|---|
| unsaturated | acrylic acid (2-propenoic acid)-CH₂═CHCOOH, used in polymer |
| monocarboxylic acids | synthesis |
| Fatty acids | medium to long-chain saturated and unsaturated monocarboxylic acids, |
| | with even number of carbons examples docosahexaenoic |
| | acid and eicosapentaenoic acid (nutritional supplements) |
| Amino acids | the building-blocks of proteins |
| Keto acids | acids of biochemical significance that contain a ketone group, |
| | e.g. acetoacetic acid and pyruvic acid |
| Aromatic carboxylic | benzoic acid, the sodium salt of benzoic acid is used as a food |
| acids | preservative, salicylic acid-a beta hydroxy type found in many skin-care |
| | products, phenyl alkanoic acids the class of compounds where a phenyl |
| | group is attached to a carboxylic acid. |
| Dicarboxylic acids | containing two carboxyl groups examples adipic acid the monomer |
| | used to produce nylon and aldaric acid-a family of sugar acids |
| Tricarboxylic acids | containing three carboxyl groups example citric acid-found in citrus |
| | fruits and isocitric acid |
| Alpha hydroxy acids | containing a hydroxy group example glyceric acid, glycolic acid and |
| | lactic acid (2-hydroxypropanoic acid)-found in sour milk; tartaric |
| | acid-found in wine |
| Divinylether fatty | containing a doubly unsaturated carbon chain attached via an ether |
| acids | bond to a fatty acid, found in some plants |

It is believed that other similar types of acids such as malic acid may also be useful in this context.

A second constituent of the composition may include a USP grade glycol that may function as a humectant. A polyethylene glycol is a preferred glycol and is preferred at about 7-25 weight percent, and more preferably at about 10-20 weight percent of the total composition.

Other glycols contemplated for use in the present invention include propane-1, 2-diol, or alpha propylene glycol, used in the food and medicine industry.

A third constituent of the composition may include an iron chloride hexahydrate (or other suitable metal chloride hydrate containing at least one metal selected from potassium, calcium, or magnesium provided at about 2-15 weight percent of the total composition.

A fourth constituent of the composition may include purified water (e.g. having CAS# [7732-18-5] provided at about 55-65 weight percent of the total composition. This amount of water may vary as other constituents vary in the composition.

A fifth constituent of the composition may include a surfactant such as Poloxamer 407, also known as Poly(ethylene glycol)-block-poly(propylene glycol)-block-poly(ethylene glycol), and commercially available by companies such as Sigma-Aldrich, for example; the composition is provided at about 7-25 weight percent, and more preferably at about 10-20 weight percent of the total composition. Other suitable surfactants or hydrophilic polymer chains such as synthetic or natural colloidal hydrogels, which may contain 90% water may also be used in the compositions of the present invention. A preferred composition contains about 18 weight percent of this constituent whereby this amount typically causes the composition to be a gel at body temperature (about 37 degrees Celsius). Iteratively determining this key point, depending on the amount of the other constituents, aids in defining the thermal transition point of the developed solution. Accordingly, it will be appreciated that the first component is preferably a liquid at room temperature, and that upon exposure to the heat attendant to the mouth of a patient, it becomes gelatinous or more viscous, thereby forming a gelatinous film on the teeth of the patient. The liquid and flowable nature of the first component makes it amenable to rapid application to the teeth of the patient, and furthermore actually more efficiently covers more of the tooth surface as compared to painting on a fluoride varnish, for example. A preferred surfactant, such as Poloxamer 407, is a hydrophilic non-ionic surfactant of the more general class of copolymers known as poloxamers. Poloxamer 407 is a triblock copolymer consisting of a central hydrophobic block of polypropylene glycol flanked by two hydrophilic blocks of polyethylene glycol.

In accordance with a second aspect of the present invention, a second component may be used in conjunction with the first component to provide an enhanced cleaning of the calculus. The second component may contain a metal carbonate in powder form, such as calcium carbonate, sodium bicarbonate, potassium bicarbonate, caesium bicarbonate, magnesium bicarbonate, calcium bicarbonate, ammonium bicarbonate—each of these when selected are provided as a solid powder, and may be provided at about a 3:10 mass ratio to the first component, and is employed as described in Example 3 below.

From a molar standpoint, it is believed that a ratio of about 2:3 of supplement or second component to citric acid may be preferably provided, to form carbon dioxide and the associated foaming. Alkaline earth metal carbonates are preferred when providing a metal carbonate.

### Example 1: Formulation of Component One

A first formulation of component one was formed by providing purified water at about 592 grams (CAS # [7732-18-5] into a mixing bowl. Anhydrous citric acid (CAS # [77-92-9] at about 100 grams was then added to the mixing bowl. Iron chloride hexahydrate (CAS # [10025-77-1] at about 30 grams was then added to the mixing bowl. Polyethylene Glycol MW 400 (CAS # [25322-68-3] at about 100 grams was then added to the mixing vessel. As with the other examples, the mixing bowl or vessel should be formed from a dense smooth-walled polycarbonate plastic or from smooth-walled glass. Metal containers and mixer components must be avoided to minimize the likelihood of potential reaction with the constituents of the composition and the metal container.

These contents were then mixed by utilizing an exemplary and commercially available mixer rated at 1.5 HP at 3000 RPM, with a preferred clearance height of 16 inches. The exemplary mixer contains plastic product-contact parts and contains a four-inch plastic dispersion blade/impeller. The variable speeds for the mixer range from 680-1200 RPM. The mixing is generally performed at ambient temperatures of 15 to 27 degrees Celsius.

Mixing is preferably initiated at the lowest setting of 680 RPM. Once the stirring was stabilized at the lowest setting, the speed was increased to about 1000 RPM. Once the stirring was stabilized at 1000 RPM, Poloxamer 407 at about 178 grams was slowly added to the mixing vessel in a sifting method until all of the material had been added. After adding the Poloxamer 407 (CAS # [9003-11-6], the mixture was stirred for about 35 minutes. After stirring, the mixture was transferred to a non-metal storage container and allowed to de-foam for a period of about twenty-four hours.

### Example 2: Formulation of Component One

A second formulation of component one was formed by providing purified water at about 570 grams (CAS # [7732-18-5] into a glass mixing container. Anhydrous citric acid (CAS # [77-92-9] at about 100 grams was then added to the glass mixing container. Iron chloride hexahydrate (CAS # [10025-77-1] at about 30 grams was then added to the glass mixing container. Polyethylene Glycol MW 400 (CAS # [25322-68-3] at about 200 grams was then added to the glass mixing container.

These contents were then mixed by utilizing an exemplary and commercially available mixer rated at 1.5 HP at 3000 RPM, with a preferred clearance height of 16 inches. The exemplary mixer contains plastic product-contact parts and contains a four-inch plastic dispersion blade/impeller. The variable speeds for the mixer range from 680-1200 RPM. The mixing is generally performed at ambient temperatures of 15 to 27 degrees Celsius.

Mixing was preferably initiated at the lowest setting of 680 RPM. Once the stirring was stabilized at the lowest setting, the speed was increased to about 1000 RPM. Once the stirring was stabilized at 1000 RPM, Poloxamer 407 at about 100 grams was slowly added to the mixing vessel in a sifting method until all of the material has been added. After adding the Poloxamer 407 (CAS # [9003-11-6], the mixture was stirred for about 35 minutes. After stirring, the mixture was transferred to a non-metal storage container and allowed to de-foam for a period of about twenty-four hours. Alternatively, the bubbles in the foam may be gently removed by placing the mixing container and agent into a laboratory device, which creates a slight vacuum atmosphere. As the mixture is subjected to the vacuum, the bubbles are ruptured thereby providing a clear mix of the agent.

### Example 3: Method of Application

1. The tooth is isolated and the site prepared in a conservative manner. The term “conservative” indicates: a) that the tooth is isolated to secure an operative field that is free of saliva, food debris, and any loose aggregates or bits of debris that may have become lodged between the teeth or even packed into any tooth cavities; b) that the patient may also be required to brush their teeth to remove any duterus that remains; and c) more generally, that the intended operative sight is clean of all debris and then optionally isolated with cotton rolls or sterile gauze.
2. Dispense the solution of component one as described in Example 1 or 2 onto a Dappen dish or small mixing bowl by gently squeezing until several drops are produced.
3. Prepare an applicator such as a cotton swab or micro brush by filling or absorbing the applicator with the appropriate amount of the solution (component one) to be applied to the calculus on the surface of the tooth.
4. Rub the solution, component one, onto the tooth surface and let sit for about twenty seconds. If desired, the clinician may then use a descaling device of their choice to loosen the attached mineralized debris that has become affixed to the tooth surfaces, and then rinse with suction to remove debris. If a second application for cleansing is considered necessary by the clinician, then a second application of the cleansing agent may be placed and any remaining debris then instrumented and again rinsed and evacuated by suction.
5. If also using the supplemental powder, component two—made from metal carbonate such as sodium carbonate or sodium bicarbonate, for example, dispense a small amount into a second Dappen dish or mixing bowl, using a tapping and rolling action into the Dappen dish or mixing bowl. Alternatively, the supplemental powder may be scooped from its packaging and placed in a second Dappen dish for easy access. This step is optional and may be desired to more aggressively cleanse the intended area of debris. The earthy powder/component two is picked up on a carrying device of a fine micro brush or similar device, and then applied to carry the powder to react with the previously applied product, thereby creating a cleansing foaming effect.
6. Component two may then be applied to the applicator and then to the tooth with a rubbing motion. If the supplemental powder (component two) does not readily adhere to the applicator, reapply as stated above. The rubbing action will produce a cleansing foaming or effervescing effect.
7. Rinse the area thoroughly for at least five seconds thereby removing substantially all of the product and evacuating the remaining debris. An evacuation device or suction means is preferably used to remove the debris that fills the foamed agent.

All chemical constituents are commercially available and may be provided as off-the-shelf products by companies such as Sigma-Aldrich or Fisher Chemical, for example. It should be understood that the preceding is merely a detailed description of various embodiments of this invention and that numerous changes to the disclosed embodiments can be made in accordance with the disclosure herein without departing from the spirit or scope of the invention, as stated in the claims appended hereto.

What is claimed is:

1. A dental cleaning system containing a composition comprising:
an anhydrous carboxylic acid;
a glycol;
a metal chloride hydrate;
water; and
a surfactant.

2. The system of claim 1 wherein said carboxylic acid is selected from citric acid, carbonic acid, formic acid, acetic acid propionic acid, butyric acid, valeric acid, caprolic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachlidic acid, unsaturated monocarboxylic acids, fatty acids, amino acids, keto acids, aromatic aarboxylic acids, dicarboxylic acids, tricarboxylic acids, alpha hydroxyl acids, divinylether fatty acids, and mixtures thereof.

3. The system of claim 1 wherein said glycol is selected from polyethylene glycol, propane-1,2-diol, alpha propylene glycol, and mixtures thereof.

4. The system of claim 1 wherein said metal chloride hydrate is selected from metal chloride hydrates containing iron, potassium, calcium, magnesium, and combinations thereof.

5. The system of claim 1 wherein said surfactant is selected from hydrophilic non-ionic surfactants.

6. The system of claim 1 wherein said carboxylic acid is provided in an anhydrous state at about 7.0-13.0 weight percent of the composition.

7. The system of claim 1 wherein said glycol is provided at about 10.0-20.0 weight percent of the composition.

8. The system of claim 1 wherein said metal chloride hydrate is provided at about 2.0-5.0 weight percent of the composition.

9. The system of claim 1 wherein said water is provided at about 55.0-65.0 weight percent of the composition.

10. The system of claim 1 wherein said surfactant is provided at about 10.0-20.0 weight percent of the composition.

11. The system of claim 1 wherein said composition contains purified water at about 59.2 weight percent, anhydrous citric acid at about 10.0 weight percent, iron chloride hexadyrate at about 3.0 weight percent, polyethylene glycol at about 10.0 weight percent, and Poly(ethylene glycol)-block-poly(propylene glycol)-block-poly(ethylene glycol) at about 17.8 weight percent, said weight percents taken by weight of the total composition.

12. The system of claim 1 wherein said composition contains purified water at about 57.0 weight percent, anhydrous citric acid at about 10.0 weight percent, iron chloride hexadyrate at about 3.0 weight percent, polyethylene glycol at about 20.0 weight percent, and Poly(ethylene glycol)-block-polypropylene glycol)-block-poly(ethylene glycol) at about 10.0 weight percent, said weight percents taken by weight of the total composition.

13. The system of claim 1 further comprising a second component selected from a metal carbonate, a metal bicarbonate, and mixtures thereof, wherein said second component is adapted to be combined with said composition.

14. A dental cleaning system containing a composition comprising:
a first component containing a composition, the composition comprising—
an anhydrous carboxylic acid provided at about 7-19 weight percent;
a glycol provided at about 10.0-20.0 weight percent;
a metal chloride hydrate provided at about 2.0-5.0 weight percent;
water provided at about 55.0-65.0 weight percent; and
a surfactant provided at about 10.0-20.0 weight percent; and
a second component selected from a metal carbonate, a metal bicarbonate, and mixtures thereof, said second component adapted to react with the first component when applied in combination therewith.

15. The system of claim 14 wherein:
said carboxylic acid is selected from citric acid, carbonic acid, formic acid, acetic acid propionic acid, butyric acid, valeric acid, caprolic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachlidic acid, unsaturated monocarboxylic acids, fatty acids, amino acids, keto acids, aromatic aarboxylic acids, dicarboxylic acids, tricarboxylic acids, alpha hydroxyl acids, divinylether fatty acids, and mixtures thereof;
said glycol is selected from polyethylene glycol, propane-1,2-diol, alpha propylene glycol, and mixtures thereof;
said metal chloride hydrate is selected from metal chloride hydrates containing iron, potassium, calcium, magnesium, and combinations thereof; and
said surfactant is selected from hydrophilic non-ionic surfactants.

16. A dental cleaning method containing the steps of:
providing a dental system containing—a first component containing a composition, the composition containing an anhydrous carboxylic acid provided at about 7-19 weight percent, a glycol provided at about 10.0-20.0 weight percent, a metal chloride hydrate provided at about 2.0-5.0 weight percent, water provided at about 55.0-65.0 weight percent, and a surfactant provided at about 10.0-20.0 weight percent;
preparing the site and teeth to be cleaned in a conservative manner;
applying component one over the teeth to be cleaned, to create a film; and
rinsing the first component, calculus, and/or any other debris from the teeth.

17. The method of claim 16 further providing the steps of:
providing the dental system with a second component selected from a metal carbonate, a metal bicarbonate, and mixtures thereof;
applying component two over the film created by component one;
allowing the contact of component one and component two to result in an effervescence over the teeth to be cleaned; and
rinsing component one, component two, calculus, and/or any other debris from the teeth.

18. The system of claim 16 wherein the composition of component one contains purified water at about 59.2 weight percent, anhydrous citric acid at about 10.0 weight percent, iron chloride hexadyrate at about 3.0 weight percent, polyethylene glycol at about 10.0 weight percent, and Poly(ethylene glycol)-block-poly(propylene glycol)-block-poly(ethylene glycol) at about 17.8 weight percent, said weight percents taken by weight of the total composition.

19. The system of claim 16 wherein said composition of component one contains purified water at about 57.0 weight percent, anhydrous citric acid at about 10.0 weight percent, iron chloride hexadyrate at about 3.0 weight percent, polyethylene glycol at about 20.0 weight percent, and Poly(ethylene glycol)-block-poly(propylene glycol)-block-poly(ethylene glycol) at about 10.0 weight percent, said weight percents taken by weight of the total composition.

20. The system of claim 16 wherein component two is provided as calcium carbonate or sodium bicarbonate.
